(19) United States
(12) Patent Application Publication
(10) Pub. No.: US 2019/0374480 A1
(43) Pub. Date: Dec. 12, 2019

(54) ARTICLES FOR TREATING CONCUSSION AND OTHER DISORDERS

(71) Applicant: FINE COTTON FACTORY INC., Toronto (CA)

(72) Inventor: Biren PATEL, Toronto (CA)

(73) Assignee: FINE COTTON FACTORY INC., Toronto, ON (CA)

(21) Appl. No.: 16/487,666

(22) PCT Filed: Feb. 21, 2018
(86) PCT No.: PCT/CA2018/000031

§ 371 Date: Aug. 21, 2019

Related U.S. Application Data
(60) Provisional application No. 62/461,566, filed on Feb. 21, 2017.

(51) Int. Cl. A61K 9/70; A61P 25/28; A61P 17/10; A61K 33/34

(52) U.S. Cl. CPC A61K 9/7007; A61K 33/34; A61P 17/10; A61P 25/28

(57) ABSTRACT

A method of reducing, preventing or treating concussion, acne or neuropathy, comprising having a patient in need thereof sleep in proximity to or in contact with an article containing copper. An article having anti-concussion, -acne or -neuropathy properties, the article comprising a copper.

### FIELD OF THE INVENTION

The present invention relates to articles containing copper useful for reducing or treating concussion, acne, neuropathy and other disorders.

### BACKGROUND OF THE INVENTION

Traumatic brain injury (TBI) is a major public health concern of epidemic proportions, with an annual incidence of 1.6 to 3.2 million in the United States. Mild TBI, of which concussion is a subset, is the most common form, representing nearly 75% of all TBIs [http://www.cdc.gov/TraumaticBrainInjury]. All age groups suffer concussions, from the very young to the elderly. Certain activities are more frequently associated with concussions, including athletics and military service, but they also result from general trauma caused by motor vehicle collisions, falls from height and assaults. Concussions often result in significant acute symptoms and in some individuals, long-term neurological dysfunction.

Acne vulgaris, also known as acne, is a long-term skin disease that occurs when hair follicles are clogged with dead skin cells and oil from the skin. Acne is characterized by areas of blackheads or whiteheads, pimples, greasy skin, and possible scarring. The resulting appearance can lead to anxiety and reduced self-esteem.

Many treatment options for acne are available, including lifestyle changes, medications, and medical procedures. Eating fewer simple carbohydrates (sugar) may help. Treatments include applying directly to the affected skin azelaic acid, benzoyl peroxide, and salicylic acid. Antibiotics and retinoids are available in formulations that are applied to the skin and taken by mouth for the treatment of acne. However, resistance to antibiotics may develop as a result of antibiotic therapy. Several types of birth control pills help against acne in women. Isotretinoin pills are usually reserved for severe acne due to greater potential side effects. Early and aggressive treatment of acne is advocated by some to lessen the overall long-term impact to individuals.

Acne was estimated to affect 660 million people globally (2013), making it the 8th most common disease worldwide. Acne commonly occurs in adolescence and affects an estimated 80-90% of teenagers in the Western world. Children and adults may also be affected before and after puberty. Although acne becomes less common in adulthood, it persists in nearly half of people into their twenties and thirties and a smaller group continue to have difficulties into their forties.

Neuropathy is damage to or disease affecting nerves, which may impair sensation, movement, gland or organ function, or other aspects of health, depending on the type of nerve affected. The treatment of peripheral neuropathy varies based on the cause of the condition, and treating the underlying condition can aid in the management of neuropathy. When peripheral neuropathy results from diabetes mellitus or prediabetes, blood sugar management is key to treatment. In prediabetes in particular, strict blood sugar control can significantly alter the course of neuropathy. In peripheral neuropathy that stems from immune-mediated diseases, the underlying condition is treated with intravenous immunoglobulin or steroids. When peripheral neuropathy results from vitamin deficiencies or other disorders, those are treated as well.

There is a need for an improved method for reducing or treating concussions, acne and neuropathy that does not involve surgical or pharmacological treatment of such disorders or that can be used in combination with other therapies.

### SUMMARY OF THE INVENTION

The present invention relates to articles containing copper that aid reduce the symptoms of concussion thereby speeding the recovery from concussion. The present invention relates also to methods of reducing or treating concussion in subject in need thereof comprising the use of copper or articles containing copper by a person or subject or patient in need thereof.

In one embodiment the present invention is a method of reducing, preventing or treating concussion, comprising having a patient in need thereof be in proximity to or in contact with an article containing copper.

In another embodiment the present invention is a method of reducing, preventing or treating acne, comprising having a patient in need thereof be in proximity to or in contact with or applying to a body surface exhibiting the acne an article containing copper.

In another embodiment the present invention is a method of reducing, preventing or treating neuropathy, comprising having a patient in need thereof be in proximity to or in contact with or applying to a body surface exhibiting the neuropathy an article containing copper.

In another embodiment the present invention is a method of reducing, preventing or treating a condition selected from shingles, eczema, dandruff, atopic dermatitis, athlete's foot, and/or diabetes, the method comprising having a patient in need thereof be in proximity to or in contact with or applying to a body surface exhibiting the condition an article containing copper.

In one aspect according to any of the previous embodiments, the article containing copper is a fabric or a foam.

In another aspect according to any of the previous embodiments, the content of copper in the article by weight is between about 0.05% to 4.5%.

In another aspect according to any of the previous embodiments, the copper is in metallic form, salts or ionic form, wherein the metallic form includes particulates, alloys and oxides.

In another aspect according to any of the previous embodiments, the method comprises having the patient in need thereof sleep in proximity to or in contact with the article containing copper.

In one embodiment, the present invention is a fabric having anti-concussion, anti-acne, anti-neuropathy properties, the fabric comprising an effective amount of copper.

In another embodiment, the present invention is a use of an article containing copper for reducing, preventing or treating concussion.

In another embodiment, the present invention relates to a use of an article containing copper for reducing, preventing or treating acne.

In another embodiment, the present invention relates to a use of an article containing copper for reducing, preventing or treating neuropathy.

In another embodiment, the present invention relates to a use of an article containing copper for reducing, preventing or treating at least one of shingles, eczema, dandruff, atopic dermatitis, athlete's foot, and/or diabetes.

In one aspect of any of the previous uses, the article containing copper is a fabric or a foam.

In another aspect according to any of the previous uses, the copper content by weight is between about 0.05% to 4.5%

In another aspect according to any of the previous uses, the copper is in metallic form, salts or ionic form, wherein the metallic form includes particulates, alloys and oxides.

### BRIEF DESCRIPTION OF THE DRAWINGS

The following figures illustrate various aspects and preferred and alternative embodiments of the invention.

FIG. 1: illustration of a knitting sequence of a copper-containing fabric according to one embodiment of the present invention.

FIG. 2: illustration of a knitting sequence of a copper-containing fabric according to another embodiment of the present invention.

FIG. 3: illustration of a knitting sequence of a copper-containing fabric according to another embodiment of the present invention.

FIG. 4: illustration of a knitting sequence of a copper-containing fabric according to another embodiment of the present invention.

### DESCRIPTION OF THE INVENTION

### Definitions

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. In order to aid in the understanding and preparation of the within invention, the following illustrative, non-limiting, examples are provided.

The term “knit” may be used as a verb (to make (a fabric or garment) by interweaving yarn or thread in a series of connected loops either by hand, with knitting needles, or on a machine) or as a noun (a fabric or garment made by knitting).

“Fiber” and “fibers” refer to any slender, elongated structure that can be carded or otherwise formed into a thread. Examples of fibers include “stable fibers,” a term that is well-known in the textile art.

“Yarn” refers to a structure comprising a plurality of threads.

“Effective amount” as used herein is meant an amount of copper content in an article, high enough to significantly positively modify the condition to be treated but low enough to avoid serious side effects (at a reasonable benefit/risk ratio), within the scope of sound medical judgment.

The present invention discloses the use of copper-containing articles used for treating or reducing the symptoms of conditions such as concussion, acne, neuropathy and other disorders including psoriasis, tennis elbow and headaches. In one embodiment of the present invention the copper may be provided in textiles or fabrics with anti-concussion, anti-acne and anti-neuropathy properties. The textiles of the present invention may be used to manufacture a variety of articles of manufacture, including mattress ticking fabric, bedding (i.e. covering of a bed such as sheets, blankets, duvet), clothing, gloves, socks, welding bibs and blankets, blankets, linings, drapes, upholstery, seat covers, automobile interiors, pillow covers, eye covers, head-bands and the like.

The applicant has shown that copper contained in articles such as textiles, foams and fabrics reduce the symptoms and speed up recovery of concussion, clears acne, can be used to treat neuropathy, tennis elbow, relief of headaches. The articles of the present invention may also be used in methods for reducing, preventing or treating conditions such as shingles, eczema, dandruff, atopic dermatitis, athlete's foot, and/or diabetes. The textiles or fabrics may be manufactured with copper-containing yarns, or may be infused, embedded or impregnated with copper. Foams may be infused, embedded or impregnated with copper. The copper—may be in metallic form (e.g., particulates, alloys and oxides), salts and/or ionic form.

In another embodiment, the copper may be provided on its own. For example, a subject may sleep in the proximity of or in contact with copper or copper containing objects or articles, such as copper coins, cables with copper wires, naked copper wires, and so forth. By “in the proximity” is meant that the subject is not in direct contact with the copper, but effectively or sufficiently close to the copper to positively exert its anti-concussion/acne/neuropathy properties. The proximity may vary with the duration of the exposure, the nature of any barriers between the copper-containing article and the subject, the number of barriers between the copper-containing article and the subject, the distance between the copper and the subject or any combination thereof. For example, the copper may be restricted to the inlay of a three-layer fabric, as such there would be at least one layer of fabric between the copper and the subject. Subject to the nature of the barrier, the effective proximity to exert its heath properties will thus be the maximum distance between the copper in the article and the subject, or the maximum number of barriers between the copper and the subject which will provide the desired effect on the subject's condition, such as concussion. In another embodiment, the subject may apply to a body surface exhibiting the acne or the neuropathy, or wrap around the head (in the case of concussion) the copper-containing article of the present invention for an effective amount of time.

The copper containing articles of the present invention were found to have a therapeutic or ameliorating effects even after 1 day of the subject sleeping in the proximity or in contact with the copper-containing articles of the present invention. As such, the subject may sleep in the proximity of or in contact with or apply the copper containing article of the present invention for less than one day, one day, more than one day, one week, more than one week, one month, more than one month, a year or even more that one year.

The textile or fabric of the present invention may be made using a yarn comprising a polymer or cellulose, or re-generated cellulose, or otherwise, and copper.

The copper may be provided as a powder, liquid or alloy forms. Polymers include a compound suitable for fiber and fabric generation including a thermoplastic polymer, polyester, nylon, rayon, polyethylene (PE), polypropylene (PP), polyethylene terephthalate (PET), polyethylene terephthalate glycol (PETG), co-PET, polylactic acid (PLA), and polytrimethylene terephthalate (PTT).

In some embodiment, the yarn of the present invention may be copper-containing fiber. An example of a copper-containing fiber is described in WO/2012/088507, the content of which is incorporated herein by reference.

In another embodiment, the textiles or fabrics of the present invention may contain copper attached, coupled or connected to the fabric, or the copper may be loose. For example, the fabric may be in the form of a bag having copper inside the bag in loose form or attached to the inside walls of the bag, or copper may be attached, connected, or coupled to an existing fabric. Alternatively, the fabric may contain one or more pockets into which the copper can be contained in loose or attached form. The pockets may be designed to be in a closed or opened from. For example, the pocket may include attachment means such as buttons or Velcro® or may include a flap to open or close the pocket. This design of pockets may allow a person to insert or remove the copper from the pockets. Copper may be provided in any form as previously described. For example, the copper may be provided as a powder, liquid, alloys, filaments, wires, or in the form of coins (for example one cent coins known as pennies). The copper may be provided in metallic form, salt from or ionic form.

The fabric of the present invention may have one, two, three, four or more than four layers. A three layer fabric (with inlay), ticking fabric and/or double knit fabric (two layer without inlay), and/or a single knit construction of any kind, or a woven of any kind or content. The copper may be included any one of the layers of the fabric, and in more than one layer. For example, in a two layer fabric, the copper may be included in both layers, or in just one layer. In a three layer fabric having a top, middle and bottom layers, the copper may be included in just one of the 3 layers (in any of the top, middle or bottom layers), in 2 of the 3 layers (in any combination of two of the top, middle or bottom layers) or in all of the 3 layers.

The applicant surprisingly discovered that the benefits of copper for concussion, acne and neuropathy, can be achieved even when the copper is contained in the inlay of the fabric, i.e. not in direct contact with the subject using the fabric. Tests on certain fabrics having the copper only in the inlay have been shown not to release copper ions. As such, the results of obtained in Example 1 below, in which the copper was restricted to the inlay (i.e. not in contact with the study participant) are surprising.

In the case of a three layer fabric, yarn position on the three layer fabric may be as follows:

1) All face feeds: copper-containing fiber; all inlay feeds: polymer filaments; and/or all back feeds: polymer filaments.

2) All face feeds: polymer fiber; all inlay feeds: copper-containing fiber; and/or all back feeds: polymer filaments.

3) All face feeds: polymer filaments; all inlay feeds: polymer filaments; and/or all back feeds: copper-containing fiber.

The yarn can be used by itself or with any other known and unknown fibers. Known fibers, such as Celliant, coolmax, cellulose fibers, FR. Fibers, infrared yarn, natural or manmade fiber, either as outer layer or inner layer as long as content of Cu content by weight is between about 0.05% to 4.5% by weight. Total content of this yarn in the fabric or textile of the present invention may be anywhere between about 8.5% to about 91.2% of the total in the fabric, including 9%, 10%, 15%, 20%, 25%, 30%, 35%, 40%, 45%, 50%, 55%, 60%, 65%, 70%, 75%, 80%, 85%, 90% and any percentage there in between.

This yarn may be spun or filament and may be used on one layer or in multiple layer fabrics. The fabric can be made using known technologies, such as knitting technology, woven technology or non-woven technology.

The fabric may be made using elastane yarn (i.e. Spendex® or Lycra®).

The copper-containing fabric or textile of the present invention may be manufactured by any method known in the art. In one embodiment, the process for making the copper-containing fabric properties includes:

(a) knitting a fabric on a circular weft knitting machine (or dial selection machine, or any other such type of knitting or weaving machine as may be invented during the term of this patent) using a group of yarns consisting of various fibers or a combination thereof;

(b) introducing a copper thread or yarn to the back of the fabric, referring to the dial in any combination of other fibers; or

(c) introducing a copper thread or yarn to the face of the fabric, referring to the cylinder in any combination of other fibers; or

(d) introducing a copper thread as a filler, referring to the space between the face and the back of the fabric in any combination of other fibers.

The knitting machine may be of various diameters and gauges and a combination thereof. The thread or yarns may be of various colors, counts or deniers and any combination thereof.

The process of manufacture may also include the use of various knitted structures and a combination thereof. Full and half gauge structures are also included and a combination thereof.

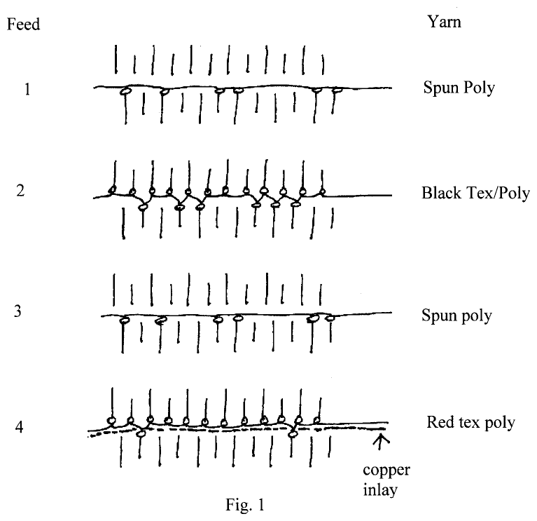

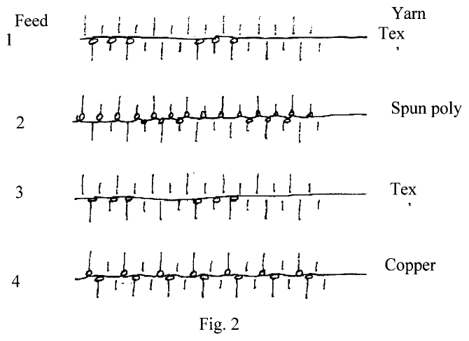

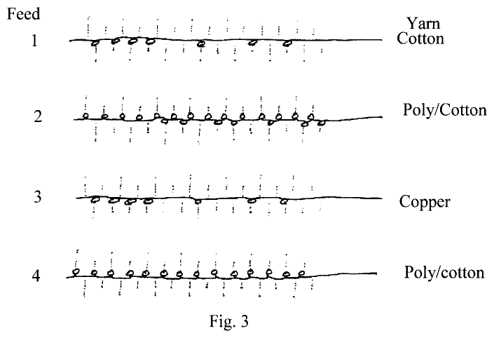

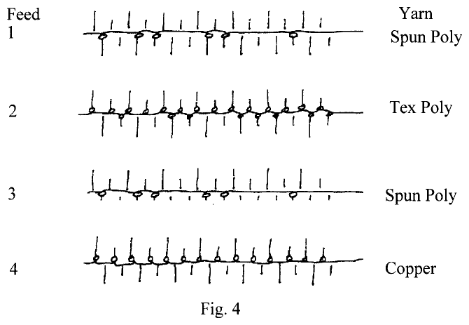

FIGS. 1-4 illustrate non-limiting knitting sequences of a copper-containing fabric according to different embodiments of the present invention.

Tables 1-4 illustrate the knitting sequences for FIGS. 1-4 respectively.

TABLE 1

| Feed # | Dial set out | Cylinder set out | Yarn |
|---|---|---|---|
| 1 | Welt all | Select as per design | Spun poly |
| 2 | Knit all | Select as per design | Black tex/Poly |
| 3 | Welt all | Select as per design | Spun Poly |
| 4 | Welt all | Select as per design | Red Tex/Poly + |
| | | | copper inlay* |

*does not knit, the copper is sandwiched between face and back of fabric

TABLE 2

| Feed # | Dial set out | Cylinder set out | Yarn |
|---|---|---|---|
| 1 | Welt all | Select as per design | Tex poly |
| 2 | Knit all | Select as per design | Spun Poly |
| 3 | Welt all | Select as per design | Tex Poly |
| 4 | Knit all | Select as per design | Copper** |

**Copper is knitting on dial and cylinder feed and showing on both face and back of fabric.

TABLE 3

| Feed # | Dial set out | Cylinder set out | Yarn |
|---|---|---|---|
| 1 | Welt all | Select as per design | Cotton |
| 2 | Knit all | Select as per design | Poly/cotton |
| 3 | Welt all | Select as per design | Copper*** |
| 4 | Knit all | Select as per design | Poly/cotton |

***Copper is knitting on cylinder only feed and showing on face only.

TABLE 4

| Feed # | Dial set out | Cylinder set out | Yarn |
|---|---|---|---|
| 1 | Welt all | Select as per design | Spun poly |
| 2 | Knit all | Select as per design | Tex/Poly |
| 3 | Welt all | Select as per design | Spun Poly |
| 4 | Knit all | Select as per design | Copper**** |

****Copper is knitting on dial only feed and showing on back only.

Alternatively, the fabric containing copper of the present invention may include a woven or non-woven textile that has been impregnated in or coated with copper. Jeffrey Gabbay et al. (J. of Industrial Textiles, vol. 35, issue 4, 2006, p. 323, the contents of which are incorporated herein by reference) provide an example on impregnating textiles with copper oxide. “Briefly, cotton fibers are plated as follows: cationic copper (a mix of Cu²⁺ and Cu¹⁺) is bound to cellulose-based fibers. Cotton, rayon/viscose, and fibers such as lyocel by Tencel are treated with an electroless plating process, which includes the following steps: (a) fibers having a diameter of about 11-13 mm are soaked for 5 s in 1% SnCl2, pH 3.5 at room temperature; (b) the fibers are then soaked for 5 s in PdCl2, pH 4 at room temperature, producing activated fibers; and (c) the activated cellulose based fibers are then exposed to formaldehyde, CuSO₄, and polyethylene glycol at pH 9. After about 5 min the fibers are plated with cationic copper (Cu(II) and Cu(I)). Finally, the fibers are dried and run through a textile carding machine which separates and aligns them.

“Impregnation of copper into the various synthetic fibers mentioned above is achieved by adding a cupric oxide powder to the polymers during the master batch preparation stage. The master batch can be made in industrially accepted concentrations and added to the polymeric slurry the same way any other master batch would be added, such as for pigmentation, etc. The copper oxide doped master batch is designed in such a way as to allow fiber extrusion in the normal production systems. The fibers can be cut into short staple or produced in filament form and texturized, if so desired. As in the case of the plated fibers mentioned above, the product yielded is a fiber that can be introduced at the blending stage of yarn production or directly into a woven or knit product so that no manufacturing processes are changed.” (See Jefferey Gabbay et al (supra) at p. 325).

The articles of the present invention, including foam, fabric or textile may have different applications, including (i) top of the bed product, (ii) mattress and mattress covers, (iii) removable mattress toppers, (iv) pillow cases, (v) pillow shell, (vi) pillow fill, (vii) blanket and blanket fill, (viii) fitted and loose sheets for bedding, (xi) mattress pads, (xii) chair liners, (xiii) sleeping bags, (xiv) clothing, including pajamas, socks and so forth, (xv) towels, (xvi) in hospital settings in mattress/toppers/removable covers/all related to mattress or any resting or sleeping surface, (xvii) in mattresses and/or (xviii) in tents. The articles of the present invention may also include items such as powders and soaps containing copper.

The articles of the present invention may be labelled for its properties. For example, the articles may include a label stating that it can reduce, prevent and/or treat one or more of concussion, acne, neuropathy, shingles, eczema, dandruff, atopic dermatitis, athlete's foot and/or diabetes.

One advantage of the copper-containing articles of the present invention is that they may be administered to a subject to treat/prevent or ameliorate a condition in a natural, non-invasive, pain-free, non-chemical, earthing way.

In order to aid in the understanding and preparation of the within invention, the following illustrative, non-limiting, examples are provided.

### Example 1

A California football player was hospitalized following a freak injury suffered in a no contact, no pads 7-on-7 contest on a Saturday.

The player received standard treatment for concussion. However, the player was not responding to the traditional treatments.

A copper-containing fabric of the present invention (34% copper spun on the face, 39% copper inlay made as a 3 layer ticking) was provided to the player and instructed to place this fabric on the bed and were instructed to sleep on it so that the body can continuously contact with the copper-containing fabric to observe changes in the symptoms of the player. This experiment was carried out for at least 100 consecutive nights.

It was found that copper-containing fabric sped the football player's recovery.

### Example 2

Neuropathy is a condition that could be relieved by treatment with copper-containing articles of the present invention.

A male participant in North Carolina had a case of neuropathy, and a copper infused fabric (92% filament yarn, single jersey) was provided to him. The individual wrapped the copper containing fabric around his hands at night. The participant expressed this treatment significantly ameliorated his suffering.

A single layer fabric of 92% infused copper was sent to the participant so he could make a pair of gloves. The participant's initial reaction was that a bigger difference was made to his neuropathy-related suffering.

### Example 3

Tennis elbow is a condition that could be relieved by treatment with copper-containing articles of the present invention.

Sleeves manufactured from copper fabric (single layer, 92% infused copper) were provided to a participant in New Jersey to treat his tennis elbow. After two days of continuous use, the participant expressed that his discomfort from this condition had been completely eliminated.

### Example 4

A sleeve as in Example 3 (i.e. (single layer, 92% copper infusion) was provided to a female participant in Ontario, Canada who was having trouble with her knee. After many visits to the doctor, the prognosis was that the cartilage was shrinking in the knee and water buildup was occurring and causing tremendous pain, so much so in fact it was a struggle for this participant to walk. After two days of wearing the sleeve over the knee, the participant expressed that the pain had totally disappeared.

### Example 5

A male participant in Ontario, Canada was getting headaches that were similar to migraines. The participant have not had such a headache since he has been sleeping on copper-containing fabric described in Example 1 (34% copper spun on the face, 39% copper inlay made as a 3 layer ticking).

### Example 6

An adult female participant with acne on her face and a pinch nerve on her left shoulder, and an adult male participant with psoriasis were provided with pillow covers and mattress covers of the present invention (34% copper spun on the face, 39% copper inlay made as a 3 layer ticking). After one month of use of the pillow and mattress covers, the participants were provided with a questionnaire.

The participants started noticing effectiveness of the fabric of the present invention within one day of use.

According to the adult female participant, the first night she found her body temperature was different. This participant states that she is usually cold all the time, however, after a first night of using the provided pillow case and mattress covers she felt no cold and she actually felt hot and had to remove her quilt.

According to the adult female participant no new acne breakouts on her face appeared after using the fabrics of the present invention.

According to the adult female participant wrapping the pillow case around her wrist helped with her pinch nerve pain.

According to two girls who slept on copper-containing pillows, within a couple weeks their acne was completely cleared up.

Through the embodiments that are illustrated and described, the currently contemplated best mode of making and using the invention is described. Without further elaboration, it is believed that one of ordinary skill in the art can, based on the description presented herein, utilize the present invention to the full extent. All publications cited are incorporated by reference.

What is claimed is:

1. A method of reducing, preventing or treating concussion, comprising having a patient in need thereof be in proximity to or in contact with an article containing copper.

2. A method of reducing, preventing or treating acne, comprising having a patient in need thereof be in proximity to or in contact with an article containing copper.

3. A method of reducing, preventing or treating neuropathy, comprising having a patient in need thereof be in proximity to or in contact with an article containing copper.

4. A method of reducing, preventing or treating a condition selected from shingles, eczema, dandruff, atopic dermatitis, athlete's foot, and diabetes, the method comprising having a patient in need thereof be in proximity to or in contact with or applying to a body surface exhibiting the condition an article containing copper.

5. The method of claim 1, 2, 3 or 4, wherein the article containing copper is a fabric or a foam.

6. The method according to any one of claims 1-5, wherein the content of copper in the article by weight is between about 0.05% to 4.5%.

7. The method according to any one of claims 1-6, wherein the copper is in metallic form, salts or ionic form, wherein the metallic form includes particulates, alloys and oxides.

8. The method according to any one of claims 1-7, wherein the method comprises having the patient in need thereof sleep in proximity to or in contact with the article containing copper.

9. A fabric having anti-concussion properties, the fabric comprising an effective amount of copper.

10. A fabric having anti-acne properties, the fabric comprising an effective amount of copper.

11. A fabric having anti-neuropathy properties, the fabric comprising an effective amount of copper.

12. A fabric having anti-eczema, dandruff, atopic dermatitis, athlete's foot, and/or anti-diabetes, properties, the fabric comprising an effective amount of copper.

13. A use of an article containing copper for reducing, preventing or treating concussion.

14. A use of an article containing copper for reducing, preventing or treating acne.

15. A use of an article containing copper for reducing, preventing or treating neuropathy.

16. A use of an article containing copper for reducing, preventing, or treating at least one of eczema, dandruff, atopic dermatitis, athlete's foot and diabetes.

17. The use of claim 13, 14, 15 or 16, wherein the article containing copper is a fabric or a foam.

18. The use according to any one of claims 13-17, wherein copper content by weight is between about 0.05% to 4.5%.

19. The use according to any one of claims 13-18, wherein the copper is in metallic form, salts or ionic form, wherein the metallic form includes particulates, alloys and oxides.
